(19) United States
(12) Patent Application Publication
(10) Pub. No.: US 2023/0072392 A1
(43) Pub. Date: Mar. 9, 2023

(54) NASAL SPRAY

(71) Applicant: BREATHE BETTER LLC, Jupiter, FL (US)

(72) Inventor: Thomas E. Lipin, Jupiter, FL (US)

(21) Appl. No.: 17/823,275

(22) Filed: Aug. 30, 2022

Related U.S. Application Data
(60) Provisional application No. 63/239,674, filed on Sep. 1, 2021.

(51) Int. Cl. A61K 31/4174; A61K 9/00; A61P 25/20; A61K 36/61; A61K 36/53

(52) U.S. Cl. CPC A61K 31/4174; A61K 9/0043; A61P 25/20; A61K 36/61; A61K 36/53

(57) ABSTRACT

A composition and method for treating, minimizing, or preventing sleep related conditions, such as snoring or sleep apnea. The composition, preferably in the form of a nasal spray, may comprise a decongestant, such as oxymetazoline, or pharmaceutically suitable salt thereof, one or more essential oil(s), and non-active ingredients. The composition is formulated for extended use, i.e. more than 5-7 days, without the risk of decongestant nasal spray rebound congestion.

### CROSS REFERENCE TO RELATED APPLICATIONS

In accordance with 37 C.F.R. 1.76, a claim of priority is included in an Application Data Sheet filed concurrently herewith. Accordingly, the present invention claims priority to U.S. Provisional Patent Application No. 63/239,674, entitled “NASAL SPRAY” filed Sep. 1, 2021. The contents of the above referenced application are incorporated herein by reference in its entirety.

### FIELD OF THE INVENTION

The present invention relates to pharmaceutical compositions for nasal administration; more particularly, pharmaceutical compositions for nasal administration to alleviate snoring.

### BACKGROUND OF THE INVENTION

Snoring is typically defined as a hoarse sound occurring when air flows past relaxed tissues in an individual's throat, causing the tissues to vibrate as the person breathes during sleep. While nearly everyone may snore occasionally, many individuals suffer from chronic snoring or sleep apnea. In addition to being a nuisance to a bed partner or other family members, chronic snoring may signal a serious health condition, such as obstructive sleep apnea (OSA). Various methods have been suggested in attempts to prevent snoring, including sleeping in particular positions, losing weight, or use of anti-snoring devices, such as breathing strips. Surgical interventions, such as tonsillectomy, laser surgery of the palate, tongue advancement procedures and nasal surgery have been used as well. However, surgical interventions often carry risks, and such procedures are not guaranteed to provide full resolution.

The initial step in treating a person that snores relates to trying to improve their nasal airway. Some of the key product types in the nasal decongestant market are sprays, tablets, capsules and strips. All of these products help to a certain degree, but even combinations rarely fully take care of the snoring problem. The most effective medications for decreasing nasal congestion are the vasoconstrictors. Vasoconstrictors, such as sympathetic amines and the imidazoles, are effective in patients in which congestion is caused by blood engorged tissue, mainly in the nasal turbinates. Upon nasal administration, vasoconstrictors cause constriction of the arterioles in the nose resulting in decreasing congestion. However, the formulations utilized in current sprays have been problematic, leading to side effects and return symptoms to a degree stronger than present initially upon discontinuation of the drug.

Steroid sprays have decongesting properties but are mainly used for allergy symptoms. One problem associated with using these types of sprays is that steroid sprays do not work on an as-needed basis. Intranasal corticosteroids take time to work. While they provide relief to allergy symptoms after 6-10 hours, relief may not be obtained for 3-6 weeks with daily use. Steroid sprays are mild sprays and rarely get rid of snoring or sleep apnea.

Embodiments of the present invention address these shortcomings and provide a composition, in the form of a nasal spray, and method for minimizing or preventing sleep related conditions or abnormalities, such as snoring or sleep apnea.

### SUMMARY OF THE INVENTION

Embodiments of the present invention relate to a composition and method for use in minimizing or preventing sleep related conditions or abnormalities, such as snoring or sleep apnea. Embodiments of the present invention may also be used to prevent or alleviate nasal problems such as sinusitis and nasal congestion, improve nasal breathing, and allow for normal nasal functions. In one embodiment, the composition, preferably formulated as a nasal spray, comprises oxymetazoline, or a pharmaceutically acceptable salt thereof, one or more essential oils, saline solution, lubricants, and buffers. The nasal spray is configured for long term use, i.e. more than 5-7 days, without suffering one or more adverse side effects typically associated with using oxymetazoline.

The nasal spray composition and method of use was designed to provide one or more of the following: improvement in nasal breathing, allow the nose to perform nasal functions (filter air, warm air, humidification, etc.), improve or stop snoring, improve sleep, provide use with no chemical addiction, minimize or eliminate known side effects related to use of oxymetazoline. Side effects include burning/stinging, bleeding, sneezing, runny nose, headaches, or nasal obstructions, with daily use.

Accordingly, it is an objective of the invention to provide a composition for use by individuals suffering from sleep related conditions or abnormalities.

It is an objective of the invention to provide a composition for use by individuals that chronically snore while sleeping.

It is a further objective of the invention to provide a method for treating, minimizing, or preventing snoring while sleeping.

It is yet another objective of the invention to provide a composition for treating snoring.

It is a still further objective of the invention to provide a composition minimizing snoring.

It is a further objective of the invention to provide a composition for preventing snoring.

It is yet another objective of the invention to provide a nasal spray for treating, minimizing, or preventing snoring while sleeping.

It is a still further objective of the invention to provide a nasal spray for treating, minimizing, or preventing snoring while sleeping which can be used for multiple days without adverse side effects.

It is a further objective of the invention to provide a nasal spray for treating, minimizing, or preventing snoring while sleeping which can be used for multiple days without suffering from a rebound congestion effect.

It is a further objective of the invention to provide a nasal spray for treating, minimizing, or preventing snoring while sleeping which can be used for more than 5-7 days without suffering from a rebound congestion effect.

It is an objective of the invention to provide a composition for use by individuals that suffer from sleep apnea.

It is a further objective of the invention to provide a method for treating, minimizing, or preventing sleep apnea.

It is yet another objective of the invention to provide a composition for treating sleep apnea.

It is a still further objective of the invention to provide a composition minimizing sleep apnea.

It is a further objective of the invention to provide a composition for preventing sleep apnea.

It is yet another objective of the invention to provide a nasal spray for treating, minimizing, or preventing sleep apnea.

It is a still further objective of the invention to provide a nasal spray for treating, minimizing, or preventing sleep apnea which can be used for multiple days without adverse side effects.

It is a further objective of the invention to provide a nasal spray for treating, minimizing, or preventing sleep apnea which can be used for multiple days without suffering from a rebound congestion effect.

It is a further objective of the invention to provide a nasal spray for treating, minimizing, or preventing sleep apnea which can be used for more than 5-7 days without suffering from a rebound congestion effect.

It is yet another objective of the invention to provide a nasal spray for improving sleeping.

It is a further objective of the invention to provide a nasal spray which minimizes or eliminates known side effects related to use of oxymetazoline, such as burning/stinging, bleeding, sneezing, runny nose, headaches, or nasal obstructions.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### DETAILED DESCRIPTION OF THE INVENTION

While the present invention is susceptible of embodiment in various forms, there is shown and will hereinafter be described a presently preferred, albeit not limiting, embodiment with the understanding that the present disclosure is to be considered an exemplification of the present invention and is not intended to limit the invention to the specific embodiments illustrated.

An embodiment of the invention provides a composition for preventing or minimizing sleep related conditions, such as snoring or sleep apnea. An embodiment of the invention further provides a method of preventing or minimizing sleep related conditions, such as snoring or sleep apnea. Preferably, the embodiment of the invention is formulated as a nasal spray with an effective amount of oxymetazoline, or pharmaceutical acceptable salts thereof. The nasal spray is configured for long term use, i.e. more than 5-7 days, without suffering one or more adverse side effects typically associated with using oxymetazoline at standard adult dosages, including rebound congestion typically seen after 5-7 days usage, or side effects such as burning/stinging, bleeding, sneezing, runny nose, headaches, or nasal obstruction.

As used herein, the term “about” or “approximately” means within an acceptable error range for the particular value as determined by one of ordinary skill in the art, which will depend in part on how the value is measured or determined, i.e., the limitations of the measurement system. For example, “about” or “approximately” can mean within 1 or more than 1 standard deviation, per the practice in the art. Alternatively, “about” or “approximately” can mean a range of up to 20%, preferably up to 10%, more preferably up to 5%, and more preferably still up to 1% of a given value. Where particular values are described in the application and claims, unless otherwise stated, the term “about” meaning within an acceptable error range for the particular value should be assumed.

As used herein, the term “effective amount” or “effective concentrations” includes a sufficient amount of the nasal spray composition, when referring to the nasal spray composition in general, to provide the desired local or system effect, preferably without adverse side effects (i.e. unwanted or adverse effects of active ingredients or the composition as a whole), or to at least partially attain the desired effect. When referring to the individual component(s) of the nasal spray composition, “effective amount” may be defined as a sufficient amount of the individual component(s) to provide its intended or desired effect.

As used herein, the term “active ingredient” or “physiologically active ingredient” may be defined as ingredients, agents, compounds, individually or in combination, which exert a physiological effect.

As used herein, the term “pharmaceutically acceptable salt(s)” may be defined as those salts of compounds of the invention that are safe and effective for topical use in mammals, and that possess the desired biological activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts.

In an embodiment of the nasal spray composition, the nasal spray composition may include effective amounts of a decongestant or vasoconstrictor, such as oxymetazoline, or pharmaceutical acceptable salts thereof, such as oxymetazoline hydrochloride. In an illustrative embodiment, the effective amount of oxymetazoline or pharmaceutical acceptable salts thereof, is less than 0.050%, between less than 0.050% and 0.010%, and more preferably, 0.025% or about 0.025%. Oxymetazoline, a member of the imidazole group, binds and activates alpha-2 adrenergic receptors. Upon nasal administration, imidazole group compounds constrict the arterioles in the nose, resulting in decreasing congestion. Concentrations of oxymetazoline (0.050%) utilized in the over-the-counter sprays have been proven to return symptoms to a degree stronger than present initially upon discontinuation of the drug. This is called the rebound effect, or rebound congestion (rhinitis medicamentosa, chemical rhinitis, or nasal spray addiction). The rebound effect may occur in as early as 3 days of use, but typically occurs in 3-5 days and sometimes 5-7 days of use. Use of oxymetazoline, or pharmaceutical acceptable salts thereof, at less than 0.050%, and preferably at 0.025%, has been determined to be effective at reducing or minimizing snoring or sleep apnea while preventing the rebound effect. As such, the nasal spray according to the present invention can be used for a time period greater than 5-7 days without the risk of suffering from any rebound effect. In use, the nasal spray was used for more than 7 days, more than 30 days, and at least six (6) months without individuals suffering from the rebound effect.

In an embodiment of the nasal spray composition, the nasal spray composition may include effective amounts of one or more essential oils, such as but not limited to tea tree oil (Melaleuca Alternifolia Leaf Oil), rosemary oil, eucalyptus oil, ginger oil, lemon oil, oregano oil, lavender oil, marjoram oil. In certain embodiments, an essential oil, such as tea tree oil, can be used at effective concentrations to do one or more of, lubricate the nose and decrease side effects, destroys germs and irritants that block airways and cause breathing difficulty, be antiseptic, anti-inflammatory, antiviral, anti-bacterial, anti-fungal, fight off yeast and other microbes, immune stimulator, reduce sinus congestion and red and itchy eyes, or reduce cold and congestion symptoms. In certain embodiments, an essential oil, such as rosemary oil, can be used at effective concentrations to do one or more of, relieve respiratory disorders, expel excess phlegm and mucous, clear the sinuses and decrease nasal congestion, lubricate the nose and decrease side effects, help fight resistant strains of bacteria that cause chronic congestion (strep pneumonia, E. coli), provide anti-fungal properties that inhibit pathogens, or disrupt fungal cell membranes. In certain embodiments, an essential oil, such as eucalyptus oil, can be used at effective concentrations to clear nasal congestion and open airways. In certain embodiments, an essential oil, such as ginger oil, can be used at effective concentrations to strengthen the immune system and fight inflammation. In certain embodiments, an essential oil, such as lemon oil, can be used at effective concentrations to help relieve cough and cold symptoms. In certain embodiments, an essential oil, such as oregano oil, can be used at effective concentrations for fighting viral infections. In certain embodiments, an essential oil, such as lavender oil, can be used at effective concentrations to sooth the respiratory tract and help with sore throat, cough and cold symptoms. In certain embodiments, an essential oil, such as marjoram oil, can be used at effective concentrations to ease respiratory disorders such as sinusitis.

In an embodiment of the nasal spray composition, the nasal spray composition may include effective amounts of a saline solution, such as a salt solution made using ANCIENT SECRETS nasal cleaning salt (a non-iodized pure salt, sodium chloride).

In an embodiment of the nasal spray composition, the nasal spray composition may include effective amounts of one or more lubricants/solvents, such as propylene glycol, polyethylene glycol.

In an embodiment of the nasal spray composition, the nasal spray composition may include effective amounts of one or more buffers, such as monobasic sodium phosphate, dibasic sodium phosphate.

In an embodiment of the nasal spray composition, the nasal spray composition may include effective amounts of one or more preservatives, such as benzalkonium chloride, benzyl alcohol, disodium edetate.

In an embodiment of the nasal spray composition, the nasal spray composition may include effective amounts of one or more viscosity increasing agents, such as povidone (polyvinylpyrrolidone).

The nasal spray composition is preferably provided in a container equipped with a pump adapted to deliver the nasal composition (preferably as a mist) to a patient's nostril. Alternatively, the container may be designed to dispense the nasal spray composition as a nasal spray.

The following are illustrative examples of the nasal spray composition:

Nasal Spray Composition: Example 1

| Component | Concentration/Amount/Range |
|---|---|
| Active Ingredient: Decongestant | Effective amounts |
| One or more essential oils | Effective amounts |
| Non-active ingredients | Effective amounts |

Nasal Spray Composition: Example 2

| Component | Concentration/Amount/Range |
|---|---|
| Decongestant: Oxymetazoline, or | <0.05% |
| pharmaceutical acceptable salts | |
| thereof | |
| Essential oil: tea tree oil | Effective amounts |
| Essential oil: rosemary oil | Effective amounts |
| Non-active ingredients | Effective amounts |

Nasal Spray Composition: Example 3

| Component | Concentration/Amount/Range |
|---|---|
| Decongestant: Oxymetazoline, or | <0.05% |
| pharmaceutical acceptable salts | |
| thereof | |
| Essential oil: tea tree oil | Effective amounts |
| Essential oil: rosemary oil | Effective amounts |
| Non-active ingredients | Effective amounts |

Nasal Spray Composition: Example 4A

| Component | Concentration/Amount/Range |
|---|---|
| Decongestant: Oxymetazoline, or | about 0.025% |
| pharmaceutical acceptable salts | |
| thereof | |
| Essential oil: tea tree oil | Effective amounts |
| Essential oil: rosemary oil | Effective amounts |
| Saline | Effective amounts |
| Lubricants | Effective amounts |
| Buffers | Effective amounts |

Nasal Spray Composition: Example 4B

| Component | Concentration/Amount/Range |
|---|---|
| Decongestant: Oxymetazoline, or | about 0.025% |
| pharmaceutical acceptable salts | |
| thereof | |
| Essential oil: tea tree oil | Effective amounts |
| Essential oil: rosemary oil | Effective amounts |
| Saline | Effective amounts |
| Lubricants | Effective amounts |
| Buffers | Effective amounts |
| Viscosity-increasing agent | Effective amounts |
| Preservative | Effective amounts |

Nasal Spray Composition: Example 5

| Component | Concentration/Amount/Range | |
|---|---|---|
| Decongestant: Oxymetazoline, or | about 0.025% | |
| pharmaceutical acceptable salts | | |
| thereof | | |
| Essential oil: tea tree oil | Effective amounts | |
| Essential oil: rosemary oil | Effective amounts | |
| Saline | 650 mg/100 | ml |
| Lubricants: polyethylene glycol | 3 ml/100 | ml |
| Lubricants: propylene glycol | 1.5 ml/100 | ml |
| Buffers: dibasic sodium phosphate | 540 mg/100 | ml |
| Buffers: monobasic sodium | 550 mg/100 | ml |

phosphate

Nasal Spray Composition: Example 6: 100 ml Solution

| Component | Concentration/Amount/Range | |
|---|---|---|
| Oxymetazoline, or pharmaceutical | 0.025 g | (25 mg) |
| acceptable salts thereof (0.025%) | | |
| Tea tree oil | 6 drops* | |
| Rosemary oil | 10 drops** | |
| Ancient Secrets Nasal Salt | 0.65 g | (650 mg) |
| Polyethylene glycol 400 | 3 | ml |
| Propylene glycol | 1.5 | ml |
| Dibasic sodium phosphate | 0.54 g | (540 mg) |
| Monobasic sodium phosphate | 0.55 g | (550 mg) |
| Benzalkonium Chloride | 0.0055 g | (5.5 mg) |
| Benzyl Alcohol | 0.5 | ml |
| Disodium Edetate | 0.05 g | (50 mg) |
| Povidone | 1 g | (1000 mg) |
| Purified water | qs | 100 ml |

*tea tree oil from Young Living, medicinal grade, 6 drops equals 0.389114 ml
**rosemary oil from Young Living, medicinal grade, 10 drops equals 0.648524 ml

In use, the method of preventing or minimizing snoring or sleep apnea may comprise providing, applying, and administrating, via nasal administration to an individual in need thereof, the nasal spray. Preferably, nasal administration is provided to individuals (human being) twelve (12) years of age and older, more preferably at least eighteen (18) years old, or older than eighteen (18) years old. In the preferred embodiment, the nasal spray, such as Example 3, comprising oxymetazoline at 0.025% with tea tree oil and rosemary oil, is used once a day.

### Initial Study

The nasal spray was provided to 136 patients suffering chronic nasal congestion and snoring and/or sleep apnea problems. All patients were instructed to administer the nasal spray once daily for a period of 30 days. All patients self-reported the following:

1. 98% of the patients in the study used the nasal spray once a day as directed.

2. When asked to rate any improvement regarding symptom improvement, a) Sleeping, b) Decreased Snoring, c) Headaches, d) Post Nasal Drip, and e) Nasal Dryness, using a rating of zero for no improvement, 1—for some improvement, 2 for a great deal of improvement, and N/A if not applicable, the patients reported:

a) Sleeping:

i. 28% said they had some improvement sleeping,

ii. 49% said they had a great deal of improvement,

iii. Accordingly, 77% of patients reported improved (some or great deal) sleeping;

b) Decreased Snoring:

i. 28% said they had some improvement of their snoring,

ii. 23% said they had a great deal of improvement of their snoring,

iii. Accordingly, 51% of patients reported improvement in snoring;

c) Headaches:

i. 18% had some improvement in their headaches,

ii. 9% had a great deal of improvement in their headaches,

iii. Accordingly, 27% of patients said the nasal spray improved their headaches;

d) Post Nasal Drip:

i. 40% had some improvement in their drainage,

ii. 24% had a great deal of improvement,

iii. Accordingly, 64% of patients stated that it improved their post nasal drip; and

e) Nasal Dryness:

i. 27% had some improvement,

ii. 23% had a great deal of improvement,

iii. Accordingly, 50% of patients said that it improved nasal dryness.

3. When asked to rate any side effects related to a) Burning and Stinging Symptoms, b) Sneezing, c) Runny Nose, and d) Headache that was noticed, using a rating of zero for none, 1 for minimal and 2 for a great deal, the patients reported:

a) Burning and Stinging:

i. 75% reported the nasal spray did not cause any burning or stinging, and

ii. 23% reported the nasal spray caused minimal burning and stinging,

iii. Therefore, 98% of patients had minimal or no burning and stinging effects;

b) Sneezing:

i. 76% reported that the spray did not make them sneeze, and

ii. 23% reported they had minimal sneezing,

iii. Therefore, 99% said they had minimal or no sneezing;

c) Runny Nose:

i. 65% reported they had no runny nose, and

ii. 8% reported they had minimal nasal drainage,

iii. Therefore, 73% said the nasal spray gave them minimal or no drainage;

d) Headache:

i. 90% reported no headache from using the spray, and

ii. 8% reported minimal headache,

iii. Therefore, 98% had minimal or no headache while using the spray.

4. When asked if the nasal spray improved their quality of life:

i. 87% of patients reported the nasal spray improved the quality of their life,

ii. 9% of patients reported perhaps, or where equivocal, the nasal spray improved the quality of their life, and

iii. 4% reported it did not improve the quality of their life.

It is to be understood that while a certain form of the invention is illustrated, it is not to be limited to the specific form or arrangement herein described. It will be apparent to those skilled in the art that various changes may be made without departing from the scope of the invention and the invention is not to be considered limited to what is described in the specification.

[symbol]

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method of reducing or eliminating a sleep related condition, comprising:
providing to an individual suffering from a sleep related condition, a nasal spray solution comprising oxymetazoline, or pharmaceutical acceptable salts thereof, at a concentration of less than 0.05%, effective amounts of one or more essential oils, and effective amounts of one or more non-active ingredients to a patient suffering from a sleep related condition.

2. The method of reducing or eliminating a sleep related condition according to claim 1, wherein said oxymetazoline, or pharmaceutical acceptable salts thereof, is provided at a concentration of between less than 0.05 and 0.01%.

3. The method of reducing or eliminating a sleep related condition according to claim 2, wherein said oxymetazoline, or pharmaceutical acceptable salts thereof, is provided at a concentration of about 0.025%.

4. The method of reducing or eliminating a sleep related condition according to claim 1, wherein said nasal spray solution is provided to an individual twelve years old, or older.

5. The method of reducing or eliminating a sleep related condition according to claim 1, wherein said non-active ingredients include a saline solution, one or more lubricants, or one or more buffers.

6. The method of reducing or eliminating a sleep related condition according to claim 1, wherein said essential oil is tea tree oil, rosemary oil, eucalyptus oil, ginger oil, lemon oil, oregano oil, lavender oil, or marjoram oil.

7. The method of reducing or eliminating a sleep related condition according to claim 6, wherein said essential oil is tea tree oil, rosemary oil, or a combination thereof.

8. The method of reducing or eliminating a sleep related condition according to claim 1, wherein said sleep related condition is chronic snoring or sleep apnea.

9. The method of reducing or eliminating a sleep related condition according to claim 1, wherein said nasal solution is administered once a day.

10. The method of reducing or eliminating a sleep related condition according to claim 1, wherein said nasal solution is administered once a day for more than 5 days.

11. The method of reducing or eliminating a sleep related condition according to claim 1, wherein said one or more lubricants is propylene glycol, polyethylene glycol, or combinations thereof.

12. The method of reducing or eliminating a sleep related condition according to claim 1, wherein said one or more buffers is monobasic sodium phosphate or dibasic sodium phosphate.

13. A pharmaceutical composition comprising:
a nasal decongestant agent, said nasal decongestant agent comprising oxymetazoline, or pharmaceutical acceptable salts thereof, at a concentration of less than 0.05%;
an effective amount of one or more essential oils; and
effective amounts of one or more non-active ingredients.

14. The pharmaceutical composition according to claim 13, further including a saline solution.

15. The pharmaceutical composition according to claim 13, wherein said effective amounts of one or more non-active ingredients comprises one or more lubricants and one or more buffers.

16. The pharmaceutical composition according to claim 13, wherein said oxymetazoline, or pharmaceutical acceptable salts thereof, is provided at a concentration of between less than 0.05 and 0.01%.

17. The pharmaceutical composition according to claim 16, wherein said oxymetazoline, or pharmaceutical acceptable salts thereof, is provided at a concentration of about 0.025%.

18. The pharmaceutical composition according to claim 13, wherein said essential oil is tea tree oil, rosemary oil, eucalyptus oil, ginger oil, lemon oil, oregano oil, lavender oil, or marjoram oil.

19. The pharmaceutical composition according to claim 18, wherein said essential oil is tea tree oil, rosemary oil, or a combination thereof.

20. The pharmaceutical composition according to claim 13, wherein said pharmaceutical composition is provided in a container equipped with a pump adapted to deliver said composition to an individual's nostril.
